(19) United States
(12) Patent Application Publication
(10) Pub. No.: US 2016/0192658 A1
(43) Pub. Date: Jul. 7, 2016

(54) HYDROGEN-CONTAINING ANTIMICROBIAL AGENT

(71) Applicants: Yoshiyuki Nasu, Aomori (JP); SHARE-X CO., LTD., Kanagawa (JP)

(72) Inventors: Yoshiyuki Nasu, Aomori (JP); Susumu Furuhashi, Kanagawa (JP)

(21) Appl. No.: 14/912,013

(22) PCT Filed: Aug. 5, 2014
(86) PCT No.: PCT/JP2014/070543

§ 371 Date: Feb. 12, 2016

(30) Foreign Application Priority Data
Aug. 13, 2013 (JP) 2013-168029

(51) Int. Cl. A01N 59/06; A01N 59/00; A61K 33/06; A61K 33/00

(52) U.S. Cl. CPC A01N 59/06; A61K 33/00; A01N 59/00; A61K 33/06

(57) ABSTRACT

An object of the present invention is to provide antimicrobial agents that do not contribute to the emergence of drug-resistant microorganisms, that present less side effects, and that can be combined with other medications while causing less adverse effects. The present invention provides antimicrobial agents containing at least one member selected from the group consisting of: at least one hydrogen isotope selected from the group consisting of hydrogen atom (H); ¹H, ²H, ³H, ⁴H, ⁵H, ⁶H and ⁷H; hydrogen molecule (H₂); a metal hydride; hydrogen ion (H⁺); hydride ion (H⁻); and atomic hydrogen. The antimicrobial agents of the present invention exhibit their antimicrobial action through the action of hydrogen, so they are less likely to induce emergence of drug-resistant microorganisms.

### FIELD

The present invention relates to antimicrobial agents. More particularly, the present invention relates to antimicrobial agents containing at least one member selected from the group consisting of: at least one hydrogen isotope selected from the group consisting of ¹H, ²H, ³H, ⁴H, ⁵H, ⁶H and ⁷H; hydrogen molecule (H₂); a metal hydride; hydrogen ion (H⁺); hydride ion (H⁻); and atomic hydrogen.

### BACKGROUND

Among microorganisms such as bacteria, fungi and viruses, pathogenic ones that infect animals or plants to cause diseases that continue doing great harm not only to the health of animals including humans but also to agricultural crops.

To cope with pathogenic microorganisms, antimicrobial agents such as antibacterial, antifungal or antiviral agents have heretofore been developed that use a variety of compounds as active ingredients.

Conventional antimicrobial agents exhibit their pharmacological actions by inhibiting the synthesis of the cell wall, cytoplasm, enzymes and other specific bacterial and fungal constituents or by inhibiting the formation or expansion of viruses. Therefore, an emergence of drug-resistant microorganisms is a problem that inevitably results from the use of these antimicrobial agents. Another concern is the side effects that may result from administering compounds that are inherently absent in the body of living organisms. A further difficulty relates to the fact that infections are often complicated by other pathological conditions; if a therapeutic for such other pathological condition is combined with a conventional antimicrobial agent, adverse effects may sometimes occur, such as attenuation of the actions of the respective drugs or enhancement of side effects.

Antimicrobial agents are therefore needed that do not contribute to the emergence of drug-resistant microorganisms, that present less side effects, and that can be combined with other medications while causing less adverse effects.

Hydrogen is the third most abundant element on Earth after oxygen and silicon, and even within the body of living organisms, it occurs abundantly in various states in which it binds to oxygen to form water, for example.

The recent finding that hydrogen in living organisms has an action for removing active oxygen species which are implicated as a factor to cause aging and life-style related diseases is attracting researchers' attention. Active oxygen species are the result of oxygen molecules in the atmosphere having changed to more reactive substances and it has been shown that when rats in which active oxygen species were generated in large amounts by temporarily arresting the cerebral bloodstream are allowed to inhale hydrogen gas, the inflammation in the brain is mitigated (Non-Patent Document 1).

Focusing on the anti-oxidation function of hydrogen, researchers are making efforts to develop hydrogen-containing materials for producing supplementary foods and food additives. For example, Patent Document 1 discloses a method for producing a coral powder which has hydrogen molecules adsorbed thereon. And Patent Document 2 discloses a method for producing edible negative hydrogen ions (H) using a feedstock containing a coral calcium powder and a wheat flour. Patent Document 3 discloses a method for producing a powder for supplementary foods which has hydrogen molecules adsorbed thereon. Patent Document 4 discloses a method for producing a reducing agent as food additive using mollusk shell and zeolite.

### CITATION LIST

### Patent Documents

Patent Document 1: WO 2009/066463 A1
Patent Document 2: JP 2005-245265 A
Patent Document 3: Japanese Patent No. 4245655
Patent Document 4: JP 2008-263941 A

### Non-Patent Documents

Non-Patent Document 1: Ohta et al., Nature Medicine 13: 688-694 (2007)

### SUMMARY

### Technical Problem

An object of the present invention is to provide antimicrobial agents that do not contribute to the emergence of drug-resistant microorganisms, that present less side effects, and that can be combined with other medications while causing less adverse effects.

### Solution to Problem

The present inventors found that hydrogen-containing compositions show high antimicrobial activity against a variety of pathogenic microorganisms. The present inventors further found that hydrogen-containing compositions also show high antimicrobial activity against a variety of drug-resistant microorganisms. The present invention has been accomplished on the basis of these findings.

Briefly, the present invention provides the following.

[1] An antimicrobial agent containing at least one member selected from the group consisting of: at least one hydrogen isotope selected from the group consisting of ¹H, ²H, ³H, ⁴H, ⁵H, ⁶H and ⁷H; a hydrogen molecule (H₂); a metal hydride; a hydrogen ion (H⁺); a hydride ion (H); and atomic hydrogen.

[2] The antimicrobial agent as recited in [1], which contains a coral powder having the hydrogen molecule adsorbed thereon.

[3] The antimicrobial agent as recited in [1] or [2], wherein the hydrogen molecule or metal hydride ionizes upon contact with water to generate a hydride ion.

[4] The antimicrobial agent as recited in [1] or [3], wherein the metal hydride is a hydride of a metal of at least one species selected from Group 1, Group 2, Group 13 or Group 14 in the periodic table of elements.

[5] The antimicrobial agent as recited in [4], wherein the metal hydride comprises calcium hydride.

[6] The antimicrobial agent as recited in any one of [1] and [3] to [5], which contains a reduction fired body of mollusk shell, livestock's bone, fish bone, calcified coral, coral calcium, calcium carbonate, silica, zeolite, or two or more combinations thereof.

[7] The antimicrobial agent as recited in any one of [1] to [6], which is for preventing or managing bacterial infection.

[8] The antimicrobial agent as recited in [7], which is for preventing or managing infection with at least one bacterium belonging to Gram-negative bacilli, Gram-negative cocci, Gram-positive cocci, or Gram-positive bacilli.

[9] The antimicrobial agent as recited in [8], wherein the bacterium is at least one bacterium selected from the group consisting of Escherichia coli, pathogenic Escherichia coli O157, Salmonella, Haemophilus influenzae, Vibrio parahaemolyticus, Enterococcus, Pneumococcus, Neisseria, Neisseria gonorrhoeae, Neisseria meningitidis, Staphylococcus aureus, Staphylococcus epidermidis, Group A Streptococcus, Group B Streptococcus, Group C/G Streptococcus, Listeria monocytogenes, Klebsiella pneumoniae, Shigella, Vibrio cholerae, B. cepacia, Citrobacter, and Serratia.

[10] The antimicrobial agent as recited in [8], wherein the bacterium is at least one bacterium selected from the group consisting of extended-spectrum β-lactamase (ESBL) producing Gram-negative bacilli, multidrug-resistant Pseudomonas aeruginosa (MDRP), New Delhi metallo-β-lactamase (NDM-1) producing Gram-negative bacilli, β-lactamase non-producing ampicillin-resistant (BLNAR) Haemophilus influenzae, methicillin-resistant Staphylococcus aureus (MRSA), vancomycin-resistant Enterococcus (VRE), penicillin-resistant Streptococcus pneumonia (PRSP), multidrug-resistant Acinetobacter (MDRA), Klebsiella pneumoniae carbapenemase producing bacterium (KPC), penicillinase-producing Neisseria gonorrhoeae (PPNG) and community-acquired infection type methicillin-resistant Staphylococcus aureus (CA-MRSA).

[11] The antimicrobial agent as recited in any one of [1] to [6], which is for preventing or managing infection with a fungus.

[12] The antimicrobial agent as recited in [11], wherein the fungus is a yeast-like fungus.

[13] The antimicrobial agent as recited in [12], wherein the yeast-like fungus is at least one yeast-like fungus selected from the group consisting of Candida albicans and Candida glabrata.

[14] The antimicrobial agent as recited in any one of [1] to [6], which is for preventing or managing infection with a virus.

[15] The antimicrobial agent as recited in [14], wherein the virus is at least one virus selected from the group consisting of viruses belonging to the family Orthomyxoviridae, viruses belonging to the family Paramyxoviridae, and viruses belonging to the family Adenoviridae.

[16] The antimicrobial agent as recited in [15], wherein the virus is at least one virus selected from the group consisting of influenza viruses, RS viruses, and adenoviruses.

[17] The antimicrobial agent as recited in [15], wherein the virus is an amantadine-resistant influenza virus or Tamiflu-resistant influenza virus.

[18] The antimicrobial agent as recited in [1], which is in a gaseous form.

[19] The antimicrobial agent as recited in any one of [1] to [17], which is in an aerosol form.

[20] The antimicrobial agent as recited in any one of [1] to [17], which is in a liquid form.

[21] The antimicrobial agent as recited in any one of [1] to [17], which is in a solid form.

[22] The antimicrobial agent as recited in [21], which is powdery.

[23] A food containing the antimicrobial agent as recited in any one of [1] to [22].

[24] A pharmaceutical composition containing the antimicrobial agent as recited in any one of [1] to [22].

[25] The pharmaceutical composition as recited in [24], which is to be used in combination with at least one other medication.

[26] A microorganism control agent containing the antimicrobial agent as recited in any one of [1] to [22].

### Advantageous Effects

The antimicrobial agents of the present invention exhibit their antimicrobial action through the action of hydrogen, so they have the advantage of being less likely to induce emergence of drug-resistant microorganisms. In addition, hydrogen, once acting on a pathogenic microorganism, binds oxygen to form water, so the antimicrobial agents of the present invention have another advantage in that they will not exert any side effects on the organism to which they are administered and cause less adverse effects even if they are used in combination with other medications.

### BRIEF DESCRIPTION OF DRAWINGS

FIG. 1 shows how a hydrogen-containing preparation effectively suppressed bacterial growth in a urine culture.

FIG. 2 shows that Staphylococcus, Streptococcus and Pneumococcus grew by different degrees between two cases of culture in a blood agar medium M-70, one where gargling with a hydrogen-containing preparation was done and one where no gargling was done.

FIG. 3 shows that Neisseria grew by different degrees between two cases of culture in a BTB lactose agar medium in, one where gargling with a hydrogen-containing preparation was done and one where no gargling was done.

FIG. 4 shows that Haemophilus influenzae grew by different degrees between two cases of culture in a blood agar medium/chocolate agar medium, one where gargling with a hydrogen-containing preparation was done and one where no gargling was done.

FIG. 5 shows that the intensity of hydrogen's antibacterial activity varies with bacterial type.

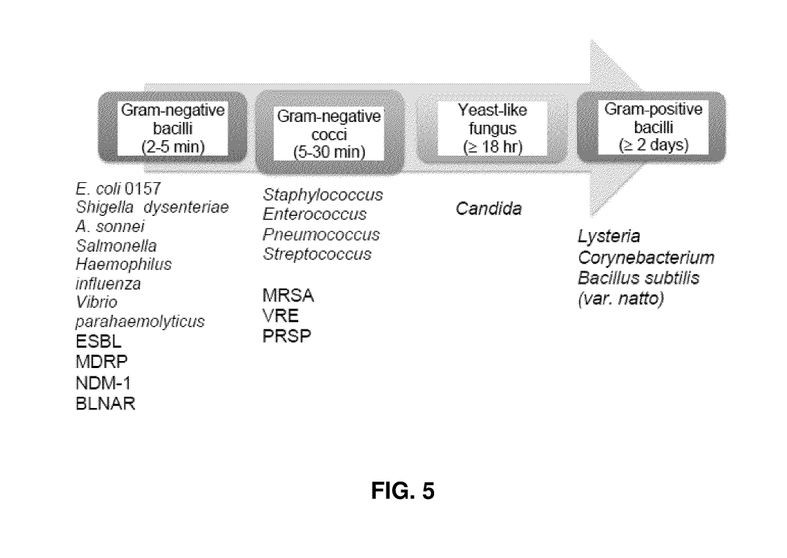

FIG. 6 is a graph showing the results of a test for evaluating hydrogen's toxicity on MDCK cells; black bars indicate the test results for hydrogen and white bars indicate the test results for physiological saline as a vehicle control; the horizontal axis of the graph plots the hydrogen concentration and the vertical axis plots the relative values with the measurements for the non-hydrogen treated group being taken as 100%; the test was conducted with N=4 and error bars indicate standard deviations.

FIG. 7 is a graph showing the results of a test for evaluating hydrogen's anti-influenza virus activity (plaque assay); gray bars indicate the test results for 5-min reaction and black bars indicate the test results for 30-min reaction; the horizontal axis of the graph plots the hydrogen concentration and the vertical axis plots the relative values for the plaques that emerged; error bars indicate standard deviations.

FIG. 8 shows the results of a test for evaluating hydrogen's anti-RS virus activity (CPE assay).

### DESCRIPTION OF EMBODIMENTS

### Hydrogen

The “hydrogen” as used herein shall refer to at least one hydrogen isotope selected from the group consisting of ¹H, ²H, ³H, ⁴H, ⁵H, ⁶H and ⁷H; a hydrogen molecule (H₂); a metal hydride; a hydrogen ion (H⁺); a hydride ion (H⁻); and atomic hydrogen.

The antimicrobial agent of the present invention contains at least one member selected from the group consisting of: at least one hydrogen isotope selected from the group consisting of ¹H, ²H, ³H, ⁴H, ⁵H, ⁶H and ⁷H; a hydrogen molecule (H₂); a metal hydride; a hydrogen ion (H⁺); a hydride ion (H⁻); and atomic hydrogen.

Hydrogen Isotopes (¹H, ²H, ³H, ⁴H, ⁵H, ⁶H and ⁷H)

Also known as light hydrogen, ¹H is the most abundant hydrogen isotope in nature and its atomic nucleus consists of only one proton; ²H is a stable hydrogen isotope having one proton and one neutron in its atomic nucleus and is also known as heavy hydrogen or deuterium; ³H has one proton and two neutrons in its atomic nucleus; ³H has one proton and two neutrons in its atomic nucleus; ⁴H has one proton and three neutrons in its atomic nucleus; ⁵H has one proton and four neutrons in its atomic nucleus; ⁶H has one proton and five neutrons in its atomic nucleus; and ⁷H has one proton and six neutrons in its atomic nucleus.

### Hydrogen Molecule (H₂)

A hydrogen molecule is represented by the molecular formula H₂. The hydrogen molecule to be used in the present invention may be of any types including, for example, a commercial product available as compressed hydrogen gas and products generated by electrolysis of water or other methods. Compressed hydrogen gas is available from, for example, Showa Denko K.K. Hydrogen molecules generated by electrolysis of water or other methods can be obtained by using, for example, the hydrogen gas generator manufactured by YMC TECHNOS CORPORATION.

The antimicrobial agent of the present invention may be adapted to contain the hydrogen gas obtained in one of the ways described above or may be so adapted as to comprise a carrier having hydrogen molecules adsorbed thereon. The carrier having hydrogen molecules adsorbed thereon that can be used may be of any types and, for example, hydrogen absorbing alloys such as AB2 type, AB5 type, Ti—Fe based, V based, Mg based, Pd based, and Ca based alloys, and coral powders having hydrogen molecules adsorbed thereon as produced by methods such as the one disclosed in WO 2009/066463 A1 can be used advantageously.

### Metal Hydrides

Metal hydrides consist of a metal element bound to a hydride ion. As the metal hydride that can be incorporated in the antimicrobial agent of the present invention, one in which an element of Group 1 (alkali metal), Group 2 (alkaline earth metal), Group 13 or Group 14 of the periodic table of elements binds to a hydride ion may be used advantageously and calcium hydride or silicon hydride can be used more advantageously.

Metal hydrides, upon contact with water, ionize to generate hydride ions (H⁻). In the water that has contacted the metal hydride, H₂, H, H⁺, H⁻, etc. are present in admixture and each of them can exhibit the antimicrobial effect.

In the antimicrobial agent of the present invention, the metal hydride may be contained on its own. Alternatively, a composition containing the metal hydride may be used as the antimicrobial agent of the present invention.

The composition containing the metal hydride refers to a composition containing not only the metal hydride but also at least one other component. The composition containing the metal hydride can be obtained by the reduction firing of an elementary metal containing composition in hydrogen gas or a gaseous mixture of hydrogen gas with an inert gas. More specifically, the metal hydride containing composition can be obtained by the reduction firing of mollusk shell, livestock's bone, fish bone, calcified coral, coral calcium, calcium carbonate, silica, zeolite, or two or more combinations thereof, for example, as an elementary metal containing composition, using methods such as the ones disclosed in JP 2005-245265 A and JP 2007-236851 A, for example. To state in greater detail, a metal hydride containing composition can advantageously be employed that has been produced by a method comprising a step in which water is added to a feedstock comprising a coral calcium powder and a wheat flour, the mixture is kneaded and then dried to make a shaped body, and a step in which the dry shaped body is oxidation fired in an oxidation firing oven for a specified period of time and thereafter reduction fired in a reducing oven in an N₂ gas/H₂ gas atmosphere for a specified period of time to make a reduction fired body. Such metal hydride containing compositions can be used on their own as the antimicrobial agents of the present invention. Alternatively, such metal hydride containing compositions may be mixed with other components to provide the antimicrobial agents of the present invention.

### Hydrogen Ion (H⁺)

The hydrogen ion is a monovalent cation that forms when a hydrogen atom loses one electron and it is also known as a hydron.

### Hydride Ion (H⁻)

The hydride ion is an anion that is represented by the ionic formula H⁻ and is also known as a hydride. The hydride ion may typically form when a hydrogen molecule cleaves in a heterolytic manner or when a metal hydride is brought into contact with water, for example. In the antimicrobial agents of the present invention, the hydride ion may be contained on its own or, alternatively, it may be contained in such a state that it can be generated from the hydrogen molecule or metal hydride.

### Atomic Hydrogen

Atomic hydrogen refers to a hydrogen atom that is generated when the covalent bond in a hydrogen molecule undergoes hemolytic cleavage (homolysis) and it is also known as a hydrogen radical or active hydrogen.

### Antimicrobial Agent

Containing at least one member selected from the group consisting of at least one hydrogen isotope selected from the group consisting of ¹H, ²H, ³H, ⁴H, ⁵H, ⁶H and ⁷H, a hydrogen molecule (H₂), a metal hydride, a hydrogen ion (H⁺), a hydride ion (H⁻) and atomic hydrogen, the antimicrobial agents of the present invention exhibit high antimicrobial activity against a wide spectrum of microorganisms including bacteria, fungi and viruses and are hence effective for the prevention or management of infection with microorganisms.

The term “prevention of infection with microorganisms” as used herein refers to enhancing the resistance of a subject against infection with microorganisms. The term “management of infection with microorganisms” as used herein refers to killing a microorganism or suppressing its expansion to thereby ameliorate the state of the subject as infected with the microorganism or inhibit the progress of the infection.

While the mechanism by which the antimicrobial agents of the present invention inactivate microorganisms is not to be bound by any theory, possible causes include, for example: damage to the cell walls or biomembranes (e.g. plasma membrane) of microorganisms due, for example, to the reducing nature of the hydrogen molecule or hydride ion or the basicity of the hydride ion; deactivation of the enzymatic activity of microorganisms; damage to the DNA of microorganisms; and impairment of the ion permeability of microorganisms. Since the antimicrobial agents of the present invention are not of such a type that they inhibit the synthesis of particular proteins by microorganisms as do the conventional antimicrobial agents, they are less likely to cause the emergence of drug-resistant microorganisms.

The bacteria to be targeted by the antimicrobial agents of the present invention are of any kinds as long as they are prokaryotic organisms having a cell membrane. In particular, bacteria belonging to Gram-negative bacilli, Gram-negative cocci, Gram-positive cocci, or Gram-positive bacilli can be advantageously targeted.

Gram-negative bacilli are a group of bacteria that are decolorized after Gram staining with the crystal violet stain to appear red or pink and the individual cells of which assume a thin rod-like or a cylindrical shape. Gram negative bacilli include, but are not limited to: Escherichia coli, pathogenic Escherichia coli O157; Salmonella enterica and Salmonella bongori; Haemophilus influenzae; Vibrio parahaemolyticus; Vibrio cholerae; Klebsiella pneumoniae; Legionella pneumophila; bacteria of the genus Pseudomonas (e.g. Pseudomonas aeruginosa), Pseudomonas syringae, Pseudomonas meliae, Pseudomonas glumae. Pseudomonas marginalis, Pseudomonas anpulliseptica, etc.); Proteus mirabilis; Serratia marcescens; Helicobacter pylori; Xanthomonas (e.g. Xanthomonas campestris, Xanthomonas citri, Xanthomonas arboricola, etc.); bacterium of the genus Acinetobacter (Acinetobacter calcoaceticus/lowffi); Shigella dysenteriae, Shigella sonnei; Yersinia enterocolitica; Stenotrophomonas maltophilia (formerly Pseudomonas maltophilia); Cepacia (Burkholderia cepacia); bacteria of the genus Citrobacter such as C. freundii, C. diversus, C. amalonaticus, etc.

Gram-negative cocci are a group of bacteria that are decolorized after Gram staining with the crystal violet stain to appear red or pink and the individual cells of which assume a spherical shape. Gram-negative cocci include, but are not limited to: bacteria of the genus Neisseria such as Neisseria gonorrhoeae, Neisseria meningitidis, etc.; and bacteria of Moraxella such as Moraxella catarrhalis, etc.

Gram-positive cocci are a group of bacteria that are not decolorized after Gram staining with the crystal violet stain to appear to be dark blue or purple-colored and the individual cells of which assume a spherical shape. Gram-negative cocci include, but are not limited to: staphylococci (bacteria of the genus Staphylococcus), enterococci (bacteria of the genus Enterococcus), and streptococci (bacteria of the genus Streptococcus such as, for example, Streptococcus pneumoniae, Group A streptococci (S. pyogenes), Group B streptococci (S. agalactiae), Group C/G streptococci (S. dysgalactiae, S. equisimilis, S. zooepidermicus, S. equi, etc.).

Gram-positive bacilli are a group of bacteria that are not decolorized after Gram staining with the crystal violet stain to appear to be dark blue or purple-colored and the individual cells of which assume a thin rod-like or a cylindrical shape. Gram-positive bacilli include, but are not limited to: bacteria of the genus Listeria; bacteria of the genus Corynebacterium; bacteria of the genus Clostridium; bacteria of the genus Bacillus; Propionibacterium acnes, etc.

The antimicrobial agents of the present invention also exhibit high antimicrobial activity against fungi. The fungi that are targeted by the antimicrobial agents of the present invention may be of any types as long as they are fungal organisms belonging to the fungus kingdom. Examples that can be targeted advantageously include: pathogens on plants such as rust fungi (fungi of the genus Puccinia), powder mildew fungi (ascomycetes belonging to the family Erysiphaceae), Aspergillus niger, Pyricularia oryzae, Rhizoctonia solani, etc.; causative fungi for mycoses on animals, such as fungi belonging to the genus Trichophyton, fungi belonging to the genus Candida, fungi belonging to the genus Cryptococcus, fungi belonging to the genus Aspergillus such as Aspergillus fumigatus, etc.

The antimicrobial agents of the present invention also exhibit high antimicrobial activity against viruses. The viruses that are targeted by the antimicrobial agents of the present invention may be of any types as long as they are structures that consist basically of a protein shell (capsid) and nucleic acids contained in it and which are capable of expansion by using another organism's cells. Examples that can be targeted advantageously include: viruses belonging to the family Poxviridae such as smallpox virus (Variola virus), cowpox virus (Variola virus), bovine papular stomatitis virus, fowlpox virus, myxoma virus, Molluscum contagiosum virus, etc.; viruses belonging to the family Herpesviridae such as simplex virus, varicella virus, lymphocryptovirus, cytomegalovirus, Roseolovirus, Rhadinovirus, etc.; viruses belonging to the family Adenoviridae including adenoviruses such as human adenovirus; viruses belonging to the family Papovaviridae such as papilomaviruses such as HPV and viruses of the genus Polyomavirus such as JCV; viruses belonging to the family Parvoviridae such as Parvovirus; viruses belonging to the family Hepadnaviridae such as Hepatitis B virus; viruses belonging to the family Arenaviridae such as Lassa virus, Tacaribe virus, Machupo virus, Junin virus, lymphocytic choriomeningitis virus, hepatitis delta virus, etc.; viruses belonging to the family Orthomyxoviridae such as Influenza virus; viruses belonging to the family Caliciviridae such as feline calicivirus, Norwalk virus, etc.; viruses belonging to the family Coronaviridae such as SARS coronavirus; viruses belonging to the family Togaviridae such as Rubella virus; viruses belonging to the family Nodavirus such as viral nervous necrosis virus; viruses belonging to the family Paramyxoviridae including RS viruses (RSV) such as mumps virus, measles virus, human respiratory syncytial virus, bovine respiratory syncytial virus, etc.; viruses belonging to the family Picornaviridae such as enterovirus, poliovirus, foot-and-mouth disease virus, etc.; viruses belonging to the family Filoviridae such as ebolavirus, etc.; viruses belonging to the family Bunyaviridae such as Crimean-Congo hemorrhagic fever virus, Tomato spotted wilt virus, etc.; viruses belonging to the family Flaviviridae such as Japanese encephalitis virus, west Nile virus, yellow fever virus, hepatitis C virus, etc.; viruses belonging to the family Rhabdoviridae such as Rabies virus, etc.; viruses belonging to the family Reoviridae such as rotavirus A; and viruses belonging to the family Retroviridae such as human immunodeficiency virus (HIV), simian immunodeficiency virus (SIV), feline immunodeficiency virus (FIV), equine infectious anemia virus (EIA), human T-lymphocytropic virus (HTLV), etc.

The antimicrobial agents of the present invention are also effective against a variety of drug-resistant microorganisms. The antimicrobial agents of the present invention also exhibit high antimicrobial activity against, for example: drug-resistant bacteria such as extended-spectrum β-lactamase (ESBL) producing Gram-negative bacilli, multidrug-resistant Pseudomonas aeruginosa (MDRP), New Delhi metallo-β-lactamase (NDM-1) producing Gram-negative bacilli, β-lactamase non-producing ampicillin-resistant (BLNAR) Haemophilus influenzae, methicillin-resistant Staphylococcus aureus (MRSA), vancomycin-resistant Enterococcus (VRE), penicillin-resistant Streptococcus pneumonia (PRSP), multidrug-resistant Acinetobacter (MDRA), Klebsiella pneumoniae carbapenemase producing bacterium (KPC), penicillinase-producing Neisseria gonorrhoeae (PPNG), community-acquired infection type methicillin-resistant Staphylococcus aureus (CA-MRSA), etc.; drug-resistant fungi such as azole-resistant Candida, etc.; and drug-resistant viruses such as drug-resistant HIV, amantadine-resistant influenza virus, Tamiflu-resistant influenza virus, etc.

What is more, since the hydrogen molecules (H₂) or hydride ions (H−) contained in the antimicrobial agents of the present invention bind oxygen to form water after they have acted on pathogenic microorganisms, the agents are less likely to cause side effects or adverse effects on the subject or environment to which they have been administered; they are also less likely to cause adverse effects even if they are used in combination with other medications.

As long as it contains at least one member selected from the group consisting of at least one hydrogen isotope selected from the group consisting of ¹H, ²H, ³H, ⁴H, ⁵H, ⁶H and ⁷H, a hydrogen molecule (H₂), a metal hydride, a hydrogen ion (H⁺), a hydride ion (H⁻) and atomic hydrogen, the antimicrobial agent of the present invention may be of any form and it can be adapted to take on a gaseous, aerosol, liquid, solid, semisolid or powdery form.

### Foods Containing the Antimicrobial Agent

The present invention also provides foods containing the above-described antimicrobial agent. The term “foods” as used herein collectively refers to ingestible solids, fluids and liquids as well as mixtures thereof.

The foods of the present invention may be of any types including, for example: beverages such as juice, milk, soft drinks, tea drinks, and tonic drinks; liquid foods such as soup; pasty foods such as jam; semi-solid foods such as jellies and gummies; solid foods such as candies, cookies, and chewing gums; oil-and-fat containing foods such as dressings and mayonnaise; pharmaceutical preparations in such forms as capsules, tablets, troches, etc.

Further examples of the foods of the present invention include, but are not limited to, nutritional supplements, health foods, functional foods, foods for young children, infant formulas, modified milk for immature infants, as well as foods and beverages for the elderly.

Nutritional supplements are foods that are enriched in particular nutrients. Health foods are healthy or health claiming foods and include nutritional supplements, natural foods, dietary foods, etc. Functional foods are foods for additionally supplying nutrients that perform body regulating functions and are synonymous with foods for specified health uses. Foods for young children are foods to be applied to children who are less than about 6 years of age. Foods for the elderly are foods that have been processed to become digestible and absorbable more easily than untreated foods. Infant formulas are modified milk for application to children who are less than about one year of age. Modified milk for immature infants is modified milk to be applied to immature infants until they are about 6 months old.

An antimicrobial agent comprising a solid carrier having hydrogen molecules adsorbed thereon or containing a metal hydride may, for example, be added to candies, chewing gums, troches or the like, whereupon they can be effectively used to prevent symptoms of common cold such as inflammation at the throat, coughing, and a runny nose, or prevent dental caries, periodontal disease, and bad breath.

If the antimicrobial agent comprising a solid carrier having hydrogen molecules adsorbed thereon or containing a metal hydride is added to tea bags or the like, there can be obtained foods in which the antiviral effect of flavonoids contained in tea leaves is synergistically combined with the antiviral effect of hydrogen.

The antimicrobial agent containing foods of the present invention may, depending on the need, include other additives such as minerals, vitamins (e.g. vitamin E, vitamin C and vitamin A), nutrients, flavors, pigments, etc. unless they impair the effects of the hydrogen isotope, hydrogen molecule (H₂), metal hydride, hydrogen ion (H⁺), hydride ion (H⁻) and atomic hydrogen, namely, unless their inclusion causes undesirable interactions with the hydrogen isotope, hydrogen molecule (H₂), metal hydride, hydrogen ion (H⁺), hydride ion (H⁻) or atomic hydrogen. Each of these additives may be of a type that is commonly used in foods and beverages. The antimicrobial agent containing foods of the present invention may also include other physiologically active ingredients unless they impair the effects of the hydrogen isotope, hydrogen molecule (H₂), metal hydride, hydrogen ion (H⁺), hydride ion (H⁻), and atomic hydrogen.

The amount of the antimicrobial agents to be contained in the foods of the present invention can be determined as appropriate for the mode and application of use. For example, if a composition containing a metal hydride is to be used as the antimicrobial agent, it may be contained in the food in an amount of 0.1 wt % to 99 wt %, preferably 1 wt % to 95 wt %, and more preferably 5 wt % to 90 wt %.

### Pharmaceutical Compositions Containing the Antimicrobial Agent

The present invention also provides pharmaceutical compositions containing the above-described antimicrobial agent. The pharmaceutical compositions of the present invention can be formulated in accordance with a specific object by common methods together with pharmacologically acceptable carriers, diluents or excipients, etc. Exemplary diluents and carriers include liquid diluents such as water, ethanol, propylene glycol, glycerin, etc. and solid diluents or excipients such as glucose, sucrose, dextrin, cyclodextrin, gum Arabic, etc. In addition, emulsifiers, tonicity agents (isotonic agents), buffers, solubilizers, antiseptics, stabilizers, antioxidants, etc. that are commonly used in formulating procedures can also be incorporated in an appropriate way.

The antimicrobial agent containing pharmaceutical compositions of the present invention may, depending on the need, include other additives such as minerals, vitamins (e.g. vitamin E, vitamin C and vitamin A), nutrients, flavors, pigments, etc. unless they impair the effects of the hydrogen isotope, hydrogen molecule (H₂), metal hydride, hydrogen ion (H⁺), hydride ion (H⁻), and atomic hydrogen, namely, unless their inclusion causes undesirable interactions with the hydrogen isotope, hydrogen molecule (H₂), metal hydride, hydrogen ion (H⁺), hydride ion (H⁻), or atomic hydrogen. Each of these additives may be of a type that is commonly used in pharmaceutical compositions.

The pharmaceutical compositions of the present invention can be used either alone or in combination with other medications.

The hydrogen isotope, hydrogen molecule (H₂), metal hydride, hydrogen ion (H⁺), hydride ion (H⁻), and atomic hydrogen as contained in the antimicrobial agents contained in the pharmaceutical compositions of the present invention have not only high antimicrobial activity but also high antioxidation activity, ATP production promoting activity and anti-inflammation activity, so even if they are used alone, they will exhibit superior therapeutic and prophylactic effects which the conventional antimicrobial agents do not have.

For example, if the pharmaceutical compositions of the present invention are applied to wounds, they suppress pathogenic microorganisms while suppressing suppuration and inflammation by virtue of their antioxidation activity and they even facilitate cell expansion by virtue of their ATP production promoting activity; as a result, they achieve a rapid wound healing effect that has been unattainable by the conventional medications.

In the case of infections with emerging microorganisms such as novel strains of pandemic influenza virus, a rapid expansion of pathogenic microorganisms and a rapid inflammatory response such as excessive cytokine generation called a cytokine storm arise as two problems. Using the pharmaceutical compositions of the present invention, however, the suppression of pathogenic microorganisms and that of inflammatory response can be accomplished at the same time.

If the pharmaceutical composition of the present invention is to be used in combination with another medication, the two may be combined in a single composition or, alternatively, they may be included in separate formulations for concurrent administration. In the case of concurrent administration, the pharmaceutical composition of the present invention may be administered prior to, or simultaneously with, or after another medication.

If the pharmaceutical composition of the present invention is used in combination with another medication, there can be obtained synergism that has been unattainable by conventional therapies.

For example, the infection with fulminant hemolytic streptococci called “flesh-eating bacteria” or vibrio vulnificus infection are serious diseases that cause soft tissue necrosis, acute renal failure, adult respiratory distress syndrome, disseminated intravascular coagulation syndrome, multiple organ failure, etc. within several tens of hours after onset, sometimes leading to a shock and even to death. However, by combining the pharmaceutical composition of the present invention with another medication, not only a treatment of the infection but also the intended organ function recovery can be achieved rapidly. Specifically, if an isotonic solution containing a penicillin antibacterial agent, an immunoglobulin preparation and the pharmaceutical composition of the present invention is administered by intravenous injection or drip infusion, the pathogenic bacterium can be killed rapidly while, at the same time, the inflammation can be effectively suppressed to prevent the disease from increasing in severity. As a further advantage, the use of conventional antibiotics can be reduced.

Speaking of urinary tract infections such as pyelonephritis, cystitis and catheter infection, their response to the treatment with conventional antimicrobial agents is satisfactory unless they are complicated by some underlying disease but if the patient has an underlying disease or is suffering from a mixed infection or an infection with a resistant microorganism, problems have arisen such as difficulty in the choice of a drug or in determining a patient-friendly therapeutic regimen. In such cases, too, the pharmaceutical composition of the present invention broadens the range over which another medication such as an antimicrobial agent to be combined can be selected, thereby enabling a more rapid treatment. In addition, treatment of an infection with a medicinal solution containing the antimicrobial agent of the present invention that is being administered through the urethra may be combined with the use of another medication to thereby enable treatment of a systemic underlying disease.

Even in the case of an infection with a drug-resistant microorganism or if the patient carriers an enzyme of their own having high activity for metabolizing a conventional antimicrobial agent, the pharmaceutical composition of the present invention may be used concurrently to achieve a synergistic therapeutic effect. For example, Helicobacter pylori is a bacterium of spiral shape that inhabits the stomach of humans, etc. and is a bacterial species that causes diseases such as peptic ulcer, duodenal ulcer and stomach cancer; the standard method for eradication of this bacterium is a one-week “triple therapy” consisting of a proton pump inhibitor (PPI), amoxicillin, and clarithromycin; however, for the reason stated above, failing cases of eradiation treatment are by no means a few. On the other hand, if the pharmaceutical composition of the present invention is concurrently used, Helicobacter pylori can be eradicated unfailingly.

If desired, fosfomycin or a salt thereof and the pharmaceutical composition of the present invention can be used in combination. As a result of this, a bimodal or multi-modal antimicrobial action is exhibited against mucoid type bacteria such as Pseudomonas aeruginosa mucoid type, Pneumococcus mucoid type, Escherichia coli mucoid type, Klebsiella pneumoniae mucoid type, Enterobacter mucoid type, etc., thus enabling more rapid and potent bacterial eradication.

Further, the pharmaceutical composition of the present invention and a variety of vaccines can be used in combination.

The medications that can be used in combination with the pharmaceutical composition of the present invention include, but are not limited to, the following examples: antibacterial agents including penicillin-type antibacterial agents such as penicillin, ampicillin, amoxicillin, methicillin, etc., β-lactamase inhibitor formulating penicillin-type antibacterial agents such as clavulanic acid/amoxicillin, etc., cephem-type antibacterial agents such as cefazolin, cefaclor, cefmetazole, cefdinir, cefepime, etc., carbapenem-type antibacterial agents such as imipenem-cilastatin, tebipenem, etc., aminoglycoside-type antibacterial agents such as kanamycin, streptomycin, neomycin, gentamicin, etc., lincomycin-type antibacterial agents such as lincomycin, clindamycin, etc., fosfomycin-type antibacterial agents such as fosfomycin, etc., tetracycline-type antibacterial agents such as tetracycline, doxycycline, minocycline, etc., chloramphenicol-type antibacterial agents such as chloramphenicol, etc., macrolide-type antibacterial agents such as erythromycin, clarithromycin, azithromycin, josamycin, etc., ketolide-type antimibacterial agents such as telithromycin, etc., polypeptide-type antibacterial agents such as bacitracin, etc., glycopeptide-type antibacterial agents such as vancomycin, etc., streptogramin-type antibacterial agents such as quinupristin-dalfopristin, etc., quinolone-type antibacterial agents such as nalidixic acid, etc., new quinolone-type antibacterial agents such as ofloxacin, levofloxacin, moxifloxacin, garenoxacin, etc., sulfa drugs such as sulfamethoxazole, trimethoprim, etc., and oxazolidinone-type antibacterial agents such as linezolid, etc.; polyene-based antibiotics such as amphotericin B, etc., fluoropyrimidine-based antibiotics such as flucytosine, etc., imidazole-based antibiotics such as miconazole, etc., triconazole-based antibiotics such as fluconazole, etc., allylamine-based antibiotics such as terbinafine hydrochloride, etc., candin-based antibiotics such as micafungin sodium, etc.; and antifungal agents such as griseofulvin, etc.; antiviral agents such as acyclovir, valacyclovir, ganciclovir, foscarnet, imiquimod, palivizumab, zanamivir, oseltamivir, peramivir, laninamivir, amantadine, T-705, azidothymidine, zidovudine, lamivudine, nevirapine, indinavir, raltegravir, maraviroc, IFNα, ribavirin, etc.; anti-inflammatory agents such as hydrocortisone, prednisolone, dexamethasone, asprin, ethenzamide, loxonin, ibuprofen, diclofenac, indomethacin, etc.; antipyretic analgesics such as acetaminophen, etc.; antihistamines such as diphenhydramine hydrochloride, chlorpheniramine maleate, epinastine hydrochloride, cimetidine, famotidine, etc.; proton pump inhibitors such as omeprazole, etc.; vaccines such as BCG, polio vaccine, measles vaccine, rubella vaccine, epidemic parotitis (mumps) vaccine, varicella vaccine, yellow fever vaccine, rotavirus vaccine, influenza virus vaccine, herpes zoster vaccine, Hib vaccine, rabies vaccine, cholera vaccine, diphtheria vaccine, pertussis vaccine, tetanus vaccine, Japanese encephalitis vaccine, pneumococcal vaccine, hepatitis A virus vaccine, hepatitis B virus vaccine, human papillomavirus vaccine, Anthrax vaccine, or combinations of two or more cholera vaccines.

The pharmaceutical compositions of the present invention may assume a gaseous, aerosol, liquid, solid, semi-solid or powdery form but these are not the sole examples.

The pharmaceutical compositions of the present invention may assume a gaseous form containing, for example, hydrogen molecules or hydride ions, or an aerosol form containing metal hydrides or hydride ions. By inhaling the pharmaceutical compositions of the present invention in a gaseous or aerosol form, microorganisms residing in the lungs or bronchi can be suppressed to prevent or treat pulmonary tuberculosis, mycoplasma infection, mycosis, bronchitis, asthma, etc.

The pharmaceutical compositions of the present invention may be adapted to be in a liquid form containing at least one member selected from the group consisting of a hydrogen isotope, a hydrogen molecule (H₂), a metal hydride, a hydrogen ion (H⁺), a hydride ion (H⁻), and atomic hydrogen. The pharmaceutical compositions of the present invention in liquid form may, for example, be used in the following applications: sprays, liniments or bath salts for preventing or managing microbial infections including Propionibacterium acnes and Trichophyton fungus residing on the skin; eye drops or ophthalmic cleaning agents for preventing or managing eye diseases including conjunctivitis, keratitis and endophthalmitis; sprays or liniments for preventing or managing otolaryngology diseases including otitis media and otitis interna; mouth washes for oral hygiene including the prevention of bad breath, dental caries, etc.; sprays, liniments or mouth washes for preventing or managing infections in dental oral surgery such as anaerobic bacterial infections; bath salts for preventing or managing skin inflammations such as atopic dermatitis; agents for preventing or managing urinary tract infections including simple urinary tract infection, complicated urinary tract infection, urethritis caused by non-catheterization, catheterization, Neisseria gonorrhoeae, Chlamydia, etc., as well as prostatitis; intravenous infusions; agents for preventing bacterial infection during dialysis; agents for treating vomitted matter from gastroenteritis due to norovirus or otherwise caused infections; additives to drinking water for pets for preventing or managing infections such as zoonosis; and disinfectants for the hands of humans including infants and young children. If desired, the pharmaceutical compositions of the present invention may be mixed with physiological saline and the like to prepare infusions that can then be introduced into the body, thereby making it possible to cope with bacteria or viruses within blood vessels or the body.

The pharmaceutical compositions of the present invention, if adapted to be in a powdery form, can be used in the following applications, for example: prevention or management of wounds such as bedsores (decubitus ulcers), cuts and abrasions by spraying the powder onto or rubbing it into the wound surface; sticking plaster or gauze with its wound healing effect enhanced by being impregnated with the powder or by spraying the powder onto or rubbing it into the wound surface contacting fabric of the sticking plaster or gauze; mycosis preventing or managing pharmaceutical compositions to be sprayed onto feet or armpits, or pharmaceutical compositions for odor control or for preventing or managing an odor associated with aging; pharmaceutical compositions for preventing or managing infections associated with female genitals such as vaginitis, vaginosis, amniotic fluid infection, internal genital infection, and external genital infection; pharmaceutical compositions for preventing or managing skin and soft tissue infections such as impetigo, pyoderma, catheter infection, and burn infection; and pharmaceutical compositions for preventing or managing food poisoning or infections in pregnant women and other patients who should not use antibiotics.

The amounts of the antimicrobial agents to be contained in the pharmaceutical compositions of the present invention can be determined as appropriate for the mode and application of use. For example, if a composition containing a metal hydride is to be used as the antimicrobial agent, it may be contained in the pharmaceutical composition in an amount of 0.1 wt % to 99 wt %, preferably 1 wt % to 95 wt %, and more preferably 5 wt % to 90 wt %.

The dosage and mode of administration of the pharmaceutical compositions of the present invention may be determined as appropriate for the subject, pathological condition and its progress, route of administration, dosage form, and other factors; for example, a composition containing a metal hydride can be administered in such a way that its amount ranges from 30 to 100 mg/kg body weight per day, preferably from 60 to 70 mg/kg body weight per day.

### Microorganism Control Composition Containing the Antimicrobial Agent

The present invention further provides microorganism control compositions containing the above-described antimicrobial agent. While the way of using the microorganism control composition is not particularly limited, it may, for example, be applied by spraying, spreading, immersing, wetting or otherwise treating the surfaces of materials, tools, agricultural crops, etc. that need be inhibited from microorganism adhesion or expansion.

The microorganism control compositions of the present invention can be prepared by being formulated into a variety of dosage forms depending on their object and use, as exemplified by liquids and solutions (including aqueous suspensions and oils), pastes, powders or dusts, granules, microcapsules, etc.

More specifically, the microorganism control compositions of the present invention can be applied such as by spraying, spreading or immersing a variety of articles for antimicrobial and disinfecting purposes, as exemplified by: hygienic products such as sticking plaster, gauze, bandage, antiseptic cotton, cotton swab, and mask; nursing care and medical equipment such as tweezers, scissors, forceps, kidney dish, stethoscope, and catheter; daily necessities such as towel, dishcloth, cutting board, kitchen knife, tableware, pot, toothbrush, clothing, and linen; and fixtures in public facilities such as washroom, door knob, hot spring, and air conditioner.

Another application of the microorganism control compositions of the present invention is for antimicrobial or disinfecting treatment of foods such as vegetables, fruits, seafood, and meats; for this purpose, the compositions may, for example, be used as a cleaning solution per se or mixed with a cleaning solution.

If a gaseous or aerosol form of the microorganism control composition of the present invention is sprayed, microorganisms in the air can be suppressed, so the composition can be applied in humidifiers or air cleaners, for example. The microorganism control composition in gaseous or aerosol form of the present invention can also be used to suppress microorganism in foods such as meats, seafood, and vegetables, so it can be applied to prevent putrefaction or oxidation of foods in homes, restaurants, factories, and the like.

Microorganism control compositions containing the antimicrobial agent comprising a solid carrier having hydrogen molecules adsorbed thereon or containing a metal hydride can, for example, be used in the following applications: including the composition in beauty packs, masks, etc. to prepare face packs having antimicrobial and beauty effects; adding the composition to soaps such as solid soap and liquid soap to prepare cleaning agents with an enhanced antimicrobial effect; using the composition as an additive that is to be added to water tanks such as for aquarium fish to suppress bacterial expansion; using the composition as an additive that is to be added to aquaculture ponds for food fish, fish preserves in restaurants, etc. to suppress bacterial expansion; adding the composition to feeds for aquarium fish, cultured fish, etc. to prevent or manage diseases; and adding the composition to feeds for livestock such as cows and pigs or poultry such as chickens to prevent or manage diseases.

The microorganism control compositions of the present invention can also be applied to agricultural crops either as a substitute for known agrichemicals or in combination therewith. As a result, the use of agrichemicals can be reduced, thereby enabling the agrichemical-induced environmental or health damage to be reduced markedly. Furthermore, if a reduction fired body of mollukshell, livestock's bone, fish bone, calcified coral, coral calcium, calcium carbonate, silica, zeolite, or two or more combinations thereof is used as the microorganism control composition of the present invention, the carrier of hydrogen molecules or hydride ions directly serves as a mineral source in the soil, which is combined with the microorganism control effect to provide a synergistic crop growth promoting effect.

Depending on their object and use, the microorganism control compositions of the present invention may contain known additives such as, for example, antifoaming agents, photostabilizers, surfactants, pH stabilizers, etc. Antifoaming agents include, but are not limited to, silicone-based antifoaming agents, fatty acid based antifoaming agents, fatty acid ester based antifoaming agents, and mineral oils. Photostabilizers include, but are not limited to, hindered amine based photostabilizers and benzoate-based photostabilizers. Surfactants include, but are not limited to, lypophilic surfactants such as triglycerides, nonionic hydrophilic surfactants such as polyethylene glycol fatty acid esters, ionic hydrophilic surfactants such as phospholipids, and mixtures thereof.

The amounts of the antimicrobial agents to be contained in the microorganism control compositions of the present invention can be determined as appropriate for the mode and application of use. For example, if a composition containing a metal hydride is to be used as the antimicrobial agent, it may be contained in the microorganism control composition in an amount of 0.1 wt % to 99 wt %, preferably 1 wt % to 95 wt %, and more preferably 5 wt % to 90 wt %.

### EXAMPLES

On the following pages, the present invention is described more specifically based on Examples. It should, however, be noted that the present invention is by no means limited to these Examples.

<Assaying the Effect of Hydrogen on Bacteria and Fungi>

### Specimens, Materials and Methods

1. Hydrogen-containing preparations: SUISO PURE (ACCHE Corporation) and SUISO Z (ACCHE Corporation)

SUISO PURE is a preparation that contains coral calcium, etc. having hydrogen molecules adsorbed thereon and which, upon contact with water, etc., generates hydrogen molecules, hydrogen ions (H⁺), hydride ions (H⁻) and atomic hydrogen (WO 2009/066463). SUISO Z is a preparation that contains coral calcium and zeolite, etc. having hydrogen molecules adsorbed thereon and which, upon contact with water, etc., generates hydrogen molecules, hydrogen ions (H⁺), hydride ions (H⁻) and atomic hydrogen.

2. Target Microbial Strains: The following eleven microbial strains were isolated from clinical materials and used in the assay: Candida albicans (yeast-like fungus), Streptococcus pneumoniae (pneumococcus), Corynebacterium striatum (positive bacillus), methicillin-resistant Staphylococcus aureus (MRSA), vancomycin-resistant enterococci (VRE), multiple drug-resistant Pseudomonas aeruginosa (MDRP), Klebsiella pneumoniae (ESBL or extended spectrum β-lactamase producing Gram-negative bacillus), Escherichia coli (O-157), Group O9 Salmonella enteritidis, Group C Shigella boydii, and Group O8 Yersinia enterocolitica. The isolated strains were identified by VITEK 2 Compact 60 (Sysmex•bioMérieux). Also used were standard strains of four bacterial species, Pseudomonas aeruginosa ATCC® 27853™, Staphylococcus aureus subsp. aureus ATCC® 29213™, Enterococcus faecalis ATCC® 29212™, and Escherichia coli ATCC® 25922™, as purchased from Micro BioLogics®.

3. Clinical Specimens: expectorated sputum and urine

4. Preparing and evaluating cell suspensions: Each of the bacterial species was cultured overnight and suspended in sterile physiological saline to give a cell density approximated by McFarland Standard No. 0.5 (ca.10⁸ CFU/ml) to thereby prepare a cell suspension as an inoculum.

Centrifuge tubes were each charged with 2 ml of sterile physiological saline, to which were added a ground hydrogen-containing preparation and 100 μl of the inoculum cell suspension, followed by mixing well with a mini-mixer. One loopful of the resulting specimen was smeared on each medium at specified intervals and cultured under aerobic conditions at 35° C. for 18-24 hours. After the culture, colony counting was done to evaluate the growth of surviving cells on the medium macroscopically on the following scale: negative (no growth); real number (1-100 cells); 1+(>10² cells); 2+(>10³ cells); 3+(>10⁴ cells).

5. Media, Materials, and Equipment: BTB lactose supplemented agar medium (Eiken Chemical Co., Ltd.), blood agar medium M-70 (Eiken Chemical Co., Ltd.), mannitol salt medium (Eiken Chemical Co., Ltd.), NAC agar medium (Eiken Chemical Co., Ltd.), D-Coccosel agar medium (Sysmex•bioMérieux), CHROMagar Candida agar medium (Japan B•D), SS agar medium (Japan B•D), CHROMagar 0157 agar medium (Japan B•D), Yersinia CIN agar medium (Sysmex•bioMérieux), Favor G stain Solution® (NISSUI PHARMACEUTICAL CO. LTD.), incubator IS900 (35° C.±1), sterile physiological saline, sterile centrifuge tube, sterile petri dish, platinum loop, gas burner, mini-mixer, and micropipette.

### Example 1

The urine of patients was cultured in a specified volume and to 1 ml of urine specimens in which more than 10⁵ cells of Escherichia coli and Klebsiella pneumoniae were mixed with a single tablet of SUISO PURE and cultured. A check was made to see whether any changes occurred in the control and after 10-min, 30-min, 1-hr, 3-hr, and 5-hr culturing. As it turned out, cell growth covered the entire medium in the control; in contrast, the overall growth decreased to about one half in the samples acted upon by SUISO PURE for 10 minutes; it decreased to one third in the samples on which SUISO PURE was acted for 30 minutes; in the samples acted upon for one hour, only a few cells survived; and the samples acted upon for 3 hours were negative.

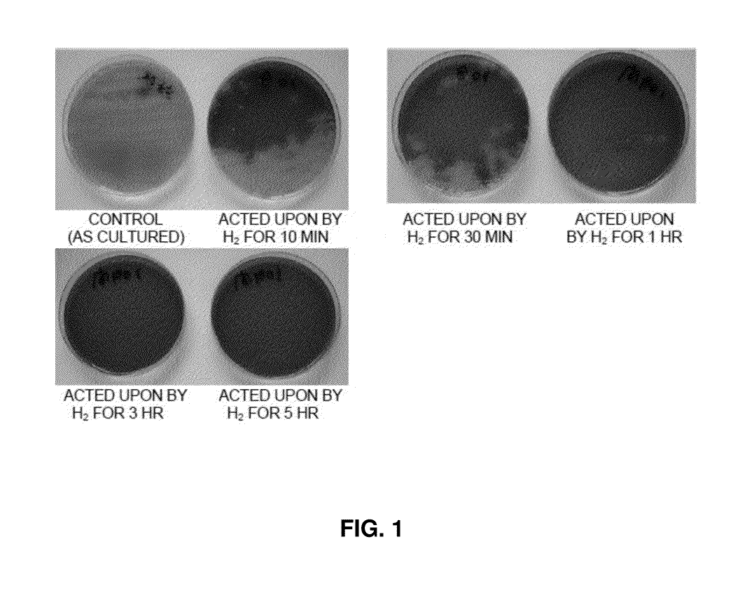

These results demonstrated that the hydrogen-containing preparations are effective in suppressing the growth of bacteria (FIG. 1).

### Example 2

The hydrogen-containing preparations were assayed for their action against various pathogenic bacteria from respiratory organs.

Sterile physiological saline (10 ml) and each of the hydrogen-containing preparations were put into the mouth, used to gargle and spat into a petri dish to prepare a sample; Sterile physiological saline (10 ml) was put into the mouth, used to gargle and spat into a petri dish to prepare another sample.

One loopful of each sample was taken and cultured in the following way.

For Streptococcus pneumoniae (S. pneumoniae), Groups A, B, C and G streptococci, as well as N. lactamica, one loopful of each sample was smeared on a blood agar medium M-70 (Eiken Chemical Co., Ltd.) and cultured under aerobic conditions at 35° C. for 18-24 hours; the results were then observed.

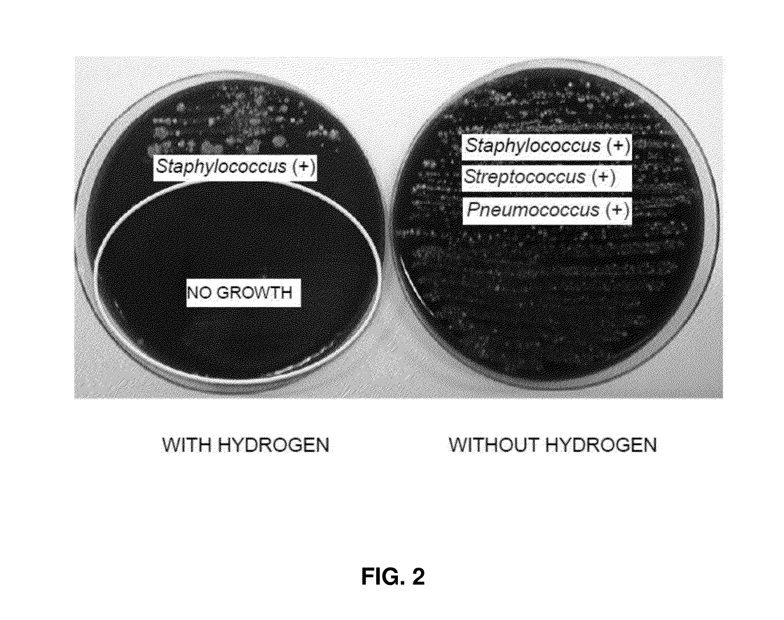

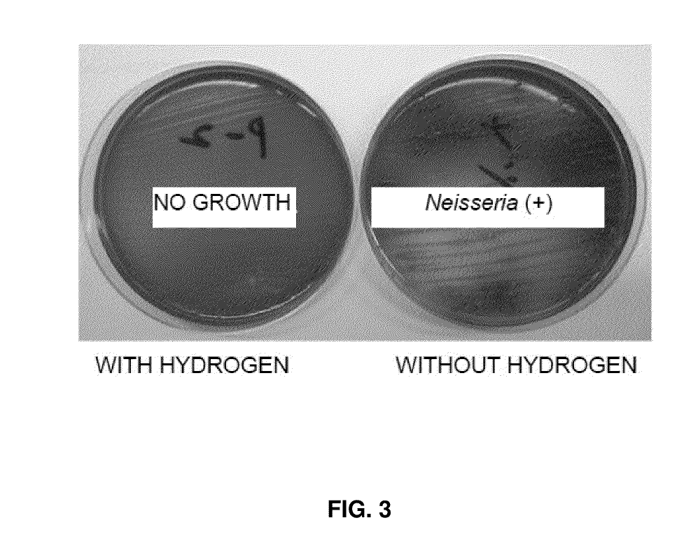

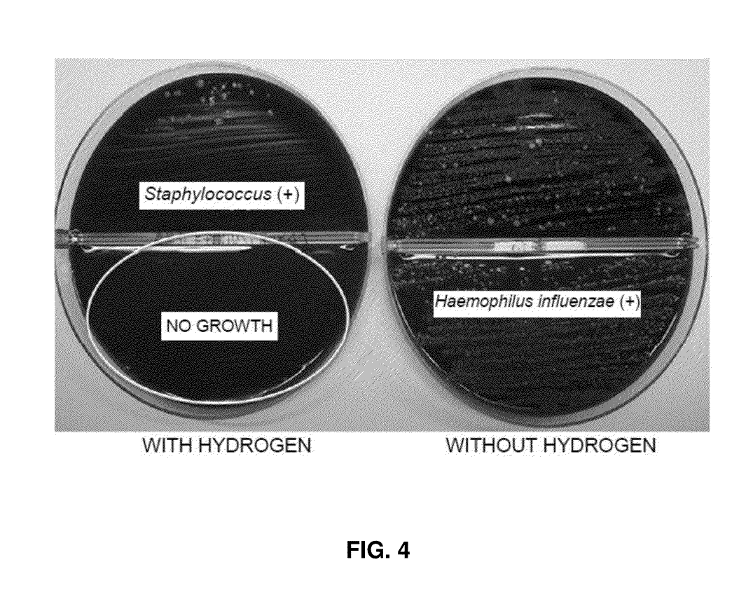

Similarly, Staphylococcus was smeared on a mannitol salt medium (Eiken Chemical Co., Ltd.), N. lactamica on a BTB lactose supplemented agar medium (Eiken Chemical Co., Ltd.) and Haemophilus influenzae on a chocolate agar medium (Eiken Chemical Co., Ltd.); the respective bacteria were cultured under aerobic conditions at 35° C. for 18-24 hours; the results were then observed. The respective results are shown in Table 1 below, and in FIGS. 2, 3 and 4.

TABLE 1
Antibacterial Effects of Hydrogen-containing Preparations against
Various Pathogenic Bacteria from Respiratory Organs

| | | | | | | | Over- |
|---|---|---|---|---|---|---|---|
| | | 5 | 10 | 15 | 30 | 60 | night |
| Hydrogen | Species Name | min | min | min | min | min | (18 hr) |
| SUISO | H. influenzae | — | — | — | — | — | — |
| Pure | S. pneumoniae | — | — | — | — | — | — |
| 2 tab. | Groups A, B, | — | — | — | — | — | — |
| | C and | | | | | | |
| | G streptococci | | | | | | |
| | N. lactamica | — | — | — | — | — | — |
| SUISO | H. influenzae | — | — | — | — | — | — |
| Z | S. pneumoniae | 3+ | 1+ | 10 | — | — | — |
| 2 tab. | | | | col- | | | |
| | | | | onies | | | |
| | Groups A, B, | 3+ | 2+ | 1+ | 3 | — | — |
| | C and | | | | col- | | |
| | G streptococci | | | | onies | | |
| | N. lactamica | 2+ | 1+ | — | — | — | — |

### Example 3

Standard strains of four bacterial species were suspended in the inoculum cell suspension, mixed with one, two or three tablets of SUISO PURE, and cultured for specified time periods, and examined closely. The standard strains were Pseudomonas aeruginosa ATCC® R27853™, Staphylococcus aureus subsp. aureus ATCC® R29213™, Enterococcus faecalis ATCC® R29212™, and Escherichia coli ATCC® R25922™. The results are shown in Tables 2, 3 and 4.

TABLE 2
Antibacterial Effects of Hydrogen-containing Preparations
against Various Standard Strains (SUISO PURE 1 tab.)

| Time | | S. aureus | E. faecalis | E. coli | P. aeruginosa |
|---|---|---|---|---|---|
| 5 | min | 3+ | 3+ | 3+ | 3+ |
| 10 | min | 3+ | 3+ | 3+ | 3+ |
| 15 | min | 3+ | 3+ | 3+ | 3+ |
| 20 | min | 3+ | 3+ | 2+ | 2+ |
| 25 | min | 2+ | 2+ | 10 colonies | 9 colonies |
| 30 | min | 2+ | 2+ | 1 colony | — |
| 35 | min | 1+ | 1+ | — | — |
| 40 | min | 1+ | 1+ | — | — |
| 45 | min | 1+ | 1+ | — | — |
| 50 | min | 1+ | 1+ | — | — |
| 55 | min | 1+ | 1+ | — | — |
| 60 | min | 10 colonies | 8 colonies | — | — |
| 65 | min | 8 colonies | 6 colonies | — | — |

| | | | | |
|---|---|---|---|---|
| overnight | — | — | — | — |

TABLE 3
Antibacterial Effects of Hydrogen-containing Preparations
against Various Standard Strains (SUISO PURE 2 tab.)

| Time | | S. aureus | E. faecalis | E. coli | P. aeruginosa |
|---|---|---|---|---|---|
| 5 | min | 3+ | 3+ | 10 colonies | 9 colonies |
| 10 | min | 2+ | 2+ | — | — |
| 15 | min | 2+ | 1+ | — | — |
| 20 | min | 2+ | 1+ | — | — |
| 25 | min | 1+ | 1+ | — | — |
| 30 | min | 1+ | 1+ | — | — |
| 35 | min | 1+ | 8 colonies | — | — |
| 40 | min | 9 colonies | 5 colonies | — | — |
| 45 | min | 3 colonies | 1 colony | — | — |
| 50 | min | — | — | — | — |
| 55 | min | — | — | — | — |
| 60 | min | — | — | — | — |
| 65 | min | — | — | — | — |

| | | | | |
|---|---|---|---|---|
| overnight | — | — | — | — |

TABLE 4
Antibacterial Effects of Hydrogen-containing Preparations
against Various Standard Strains (SUISO PURE 3 tab.)

| Time | | S. aureus | E. faecalis | E. coli | P. aeruginosa |
|---|---|---|---|---|---|
| 5 | min | 2+ | 2+ | 1 colony | 1 colony |
| 10 | min | 1+ | 1+ | — | — |
| 15 | min | 1+ | 1+ | — | — |
| 20 | min | 10 colonies | 10 colonies | — | — |
| 25 | min | 8 colonies | 9 colonies | — | — |
| 30 | min | 2 colonies | 3 colonies | — | — |
| 35 | min | — | — | — | — |
| 40 | min | — | — | — | — |
| 45 | min | — | — | — | — |
| 50 | min | — | — | — | — |
| 55 | min | — | — | — | — |
| 60 | min | — | — | — | — |
| 65 | min | — | — | — | — |

| | | | | |
|---|---|---|---|---|
| overnight | — | — | — | — |

For Staphylococcus aureus and Enterococcus faecalis, the colony counts decreased markedly in all groups administered with SUISO PURE; a decrease to 1+(11-30 colonies) occurred after 55 minutes in the one tablet group, after 30 minutes in the two tablet group, and after 15 minutes in the three tablet group.

For Pseudomonas aeruginosa and Escherichia coli, the cell growth was suppressed markedly in all groups administered with SUISO PURE; a decrease to 2+ occurred in 20 minutes in the one tablet group, and in the two tablet group or the three tablet group, the colony count decreased to 10 or less in 5 minutes.

Hence, it was then demonstrated that SUISO PURE had strong antibacterial activity against both Gram-positive and Gram-negative bacteria.

The results were the same whether the culture system was closed (using a vessel with a lid) or open (using a vessel without a lid).

It was interesting to observe that when acted upon by the hydrogen-containing preparations, the Gram-negative bacteria (Escherichia coli and Pseudomonas aeruginosa) were prone to be inactivated earlier than the Gram-positive bacteria (Staphylococcus aureus and Enterococcus faecalis). This means a great biological difference between Gram-positive and Gram-negative bacteria and the difference in the structure of the cell surface layer presumably caused the difference in growth. To be more specific, in Gram-positive bacteria, 40-70% of their outer membrane is composed of a peptidoglycan layer and the lipid content of their cell wall is low. In Gram-negative bacteria, the peptidoglycan layer is thin, accounting for only a few percent and the cell wall's lipid content is high. It is speculated that this difference in the thickness of the cell surface layer's structure would have brought the difference in the time it took for the hydrogen-containing preparations to cause cytolysis.

### Example 4

Candida albicans (yeast-like fungus) was mixed with 2 tablets of SUISO Z or SUISO PURE and cultured for specified periods of time for comparison.

Both the group administered with two tablets of SUISO Z and the group administered with two tablets of SUISO PURE saw cell growth until the lapse of 2 hours but they turned negative after the culture overnight (Table 5).

TABLE 5
Antifungal Effect of Hydrogen-Containing
Preparations against Candida albicans

| | 5 | 10 | 15 | 20 | 30 | 60 | 120 | Over- |
|---|---|---|---|---|---|---|---|---|
| | min | min | min | min | min | min | min | night |
| SUISO | 3+ | 3+ | 3+ | 3+ | 3+ | 3+ | 3+ | — |
| PURE | | | | | | | | |
| 2 tab. | | | | | | | | |
| SUISO | 3+ | 3+ | 3+ | 3+ | 3+ | 3+ | 3+ | — |
| Z | | | | | | | | |
| 2 tab. | | | | | | | | |

### Example 5

The hydrogen-containing preparations (SUISO PURE and SUISO Z) were assayed for their action against drug-resistant microorganisms. Four bacterial species, MRSA, VRE, MDRP and ESBL, were chosen as the target microorganisms.

The hydrogen-containing preparations (SUISO PURE and SUISO Z) were also assayed for their action against microorganisms associated with food poisoning. Four bacterial species, Escherichia coli O-157, Group O9 Salmonella enteritidis, Group C Shigella boydii, and Group O8 Yersinia enterocolitica, were chosen as the target microorganisms.

To 2 ml of sterile physiological saline, each of the hydrogen-containing preparations was added and after they were completely dissolved, a cell suspension of McFarland Standard No. 0.5 (ca.10⁸ CFU/ml) was added in a volume of 100 μl and mixed well. Following culture for periods of 5 min, 10 min, 15 min, 20 min, 30 min, 60 min and overnight, one loopful of the broth was smeared on a medium.

The results are shown in Tables 6 and 7.

TABLE 6
Antibacterial Effect of the Hydrogen-Containing Preparations
against Various Drug-Resistant Microorganisms

| | | | | | | | Over- |
|---|---|---|---|---|---|---|---|
| Prep- | Species | 5 | 10 | 15 | 30 | 60 | night |
| aration | Name | min | min | min | min | min | (18 hr) |
| SUISO | MRSA | 3+ | 2+ | 1+ | — | — | — |
| PURE | MDRP | — | — | — | — | — | — |
| 2 tab. | VRE | — | — | — | — | — | — |
| | ESBL | 2 col- | — | — | — | — | — |
| | | onies | | | | | |
| SUISO Z | MRSA | 3+ | 3+ | 3+ | — | — | — |
| 2 tab. | MDRP | 3+ | 2+ | — | — | — | — |
| | VRE | 1 col- | 3 col- | — | — | — | — |
| | | ony | onies | | | | |
| | ESBL | 3+ | 8 col- | — | — | — | — |
| | | | onies | | | | |

TABLE 7
Antimicrobial Effect of the Hydrogen-Containing Preparations
against Various Microorganisms Associated with Food Poisoning

| | | | | | | | Over- |
|---|---|---|---|---|---|---|---|
| | Species | 5 | 10 | 15 | 30 | 60 | night |
| Preparation | name | min | min | min | min | min | (18 hr) |
| SUISO | E. coli O157 | — | — | — | — | — | — |
| PURE | Shigella | — | — | — | — | — | — |
| 2 tab. | Salmonela | — | — | — | — | — | — |
| | Yersinia | — | — | — | — | — | — |
| SUISO | E. coli O157 | 3+ | 13 | — | — | — | — |
| Z | | | colonies | | | | |
| 2 tab. | Shigella | 3+ | 2+ | — | — | — | — |
| | Salmonela | 3+ | 2+ | — | — | — | — |
| | Yersinia | 3+ | 2+ | 1+ | — | — | — |

Regardless of the kinds of drug-resistant microorganisms and toxin-producing microorganisms, SUISO PURE acted cytolytically in a few minutes to show strong antimicrobial activity.

SUISO Z also acted cytolytically in 10 to 15 minutes, again showing strong antimicrobial activity.

### Example 6

In the same way as in Example 5, a cell suspension of Streptococcus pneumoniae (S. pneumoniae) or C. striatum (Gram-positive coccus) was mixed well with two tablets of SUISO PURE or SUISO Z and after culture for specified periods of time, one loopful of the broth was smeared on a medium.

The results are shown in Table 8.

TABLE 8
Antimicrobial Effect of the Hydrogen-Containing Preparations
against S. pneumoniae and C. striatum

| Prep- | Species | 5 | 10 | 15 | 30 | 60 | Overnight |
|---|---|---|---|---|---|---|---|
| aration | Name | min | min | min | min | min | (18 hr) |
| SUISO | S. pneumoniae | — | — | — | — | — | — |
| PURE | C. striatum | 2+ | 1 col- | — | — | — | — |
| 2 tab. | | | ony | | | | |
| SUISO Z | S. pneumoniae | 3+ | 1+ | 10 | — | — | — |
| 2 tab. | | | | col- | | | |
| | | | | onies | | | |
| | C. striatum | 3+ | 3+ | 2+ | 1+ | 8 | 5 |
| | | | | | | col- | col- |
| | | | | | | onies | onies |

In the groups administered with two tablets of SUISO PURE, S. pneumoniae and C. striatum had their growth suppressed in 5 and 15 minutes, respectively.

### Assaying the Effect Against Viruses

### Example 7

### Tests for Evaluating the Anti-Influenza Virus Activity of Hydrogen

Materials

1. Hydrogen-containing preparation: SUISO PURE GOLD (ACCHE Corporation) was used. SUISO PURE GOLD (ACCHE Corporation) is a preparation that contains coral calcium, etc. having hydrogen molecules adsorbed thereon and which, upon contact with water, etc., generates hydrogen molecules, hydrogen ions (H⁺), hydride ions (H⁻) and atomic hydrogen (WO 2009/066463). Physiological saline was used as the solvent.

2. Viral strain: A/Nagasaki/HA-58/2009 (H1 N1), the new strain of influenza virus responsible for the flu pandemic that broke out in 2009, was used. This strain has a Tamiflu-resistance mutation (H274Y). It was isolated from patient specimens and amplified using MDCK cells (to be described below).

3. Cell line: The canine kidney derived cell line, MDCK (Madin-Darby canine kidney) cell, was used. The MDCK cells were maintained in a MEM supplemented with 5% fetal bovine serum (FBS).

Test for Evaluating the Cytotoxicity of Hydrogen

1. Method of preparing a hydrogen solution

The hydrogen-containing preparation (hydrogen powder) weighing three grams (corresponding to 3 capsules) was mixed with 20 milliliters of physiological saline and the mixture was incubated at room temperature for an hour to prepare a solution containing the test substance (hydrogen) at an approximate concentration of 400 ppb (hereinafter referred to as the “hydrogen solution”).

2. Test method of assaying cytotoxicity

The MDCK cells were suspended in a MEM supplemented with 10% FBS and seeded on a 96-well plate to give a density of 3.0×10⁴ cells per well. The medium volume was adjusted to 0.1 milliliter per well.

The seeded cells were cultured at 37° C. overnight in a 5% CO₂ atmosphere. Then, the broth was removed and the monolayer of cells was washed once with a serum-free MEM (0.1 milliliter per well). Subsequently, 2-fold serial dilutions of the hydrogen solution (Table 9) were added to each well (0.2 milliliters per well) and culture was performed at 37° C. for 72 hours in a 5% CO₂ atmosphere. After the culture, live cells were stained by crystal violet staining and absorbance (OD at 560 nm) was measured with a microplate reader (Infinite M200 manufactured by TECAN).

TABLE 9

| Assay Concentrations of the Test Substance | | | | | | | | | | | | | |
|---|---|---|---|---|---|---|---|---|---|---|---|---|---|
| Assay concentrations of the test substance | | | | | | | | | | | | | |
| Plate lane | 2-fold serial dilutions = assay concentrations | | | | | | | | | | | | |
| No. | 1 | 2 | 3 | 4 | 5 | 6 | 7 | 8 | 9 | 10 | 11 | 12 | |
| Test | 0 | 0.20 | 0.39 | 0.78 | 1.56 | 3.13 | 6.25 | 12.5 | 25 | 50 | 100 | 200 | (ppb) |
| substance's | | | | | | | | | | | | | |
| concentration | | | | | | | | | | | | | |

3. Results and Discussion

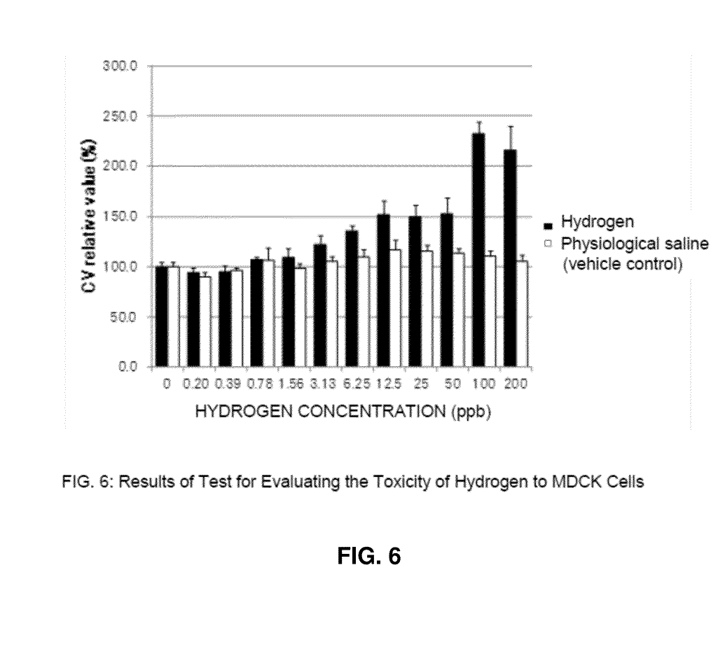

The results are shown in FIG. 6.

Hydrogen was not found to have any cytotoxicity to the MDCK cells. No marked change was found in the cells under microscopic examination.

Evaluating the Anti-Influenza Virus Activity of Hydrogen (by Plaque Reduction Test)

1. Method of preparing a hydrogen solution

A hydrogen solution was prepared by the same method as described above.

2. Method of conditioning a virus fluid

A virus fluid of the strain A/Nagasaki/HA-58/2009 (H1 N1) was diluted with a serum-free MEM for adjustment to an infectious viral titer of 4.0×10⁴ (pfu/mL).

3. Method of reaction between hydrogen and virus

The hydrogen solution was diluted with physiological saline to concentrations of 0, 40, and 400 ppb. The thus conditioned hydrogen solutions were each mixed with an equal volume of the virus fluid conditioned in 2 above; after reaction at 37° C. for 5-30 minutes (Table 10), each reaction fluid was immediately diluted 10-, 100- or 1000-fold with a serum-free MEM and subjected to a plaque forming test.

TABLE 10

| | Test Group No. | | | | | | |
|---|---|---|---|---|---|---|---|
| | #0-5 | #20-5 | #200-5 | #0-30 | #20-30 | #200-30 | |
| 400 ppb H₂ solution | 0 | 10 | 100 | 0 | 10 | 100 | μL |
| (solvent: | | | | | | | |
| physiological saline) | | | | | | | |
| Physiological saline | 100 | 90 | 0 | 100 | 90 | 0 | μL |
| Virus fluid (4.0 × 10⁴ | 100 | 100 | 100 | 100 | 100 | 100 | μL |
| pfu/mL) | | | | | | | |
| Total fluid volume | 200 | 200 | 200 | 200 | 200 | 200 | μL |
| Test substance's | 0 | 20 | 200 | 0 | 20 | 200 | |
| concentration (ppb) | | | | | | | |

| | | |
|---|---|---|
| Reaction time | 5 min. | 30 min. |

| | |
|---|---|
| Reaction temperature | 37° C.^{N.B.} |
| N number | 2 (duplicate) |

N.B.: A thermostated incubator was used.
Total number of groups = 12 groups

4. Method of calculating viral titer: Plaque assay

4.1. Method of diluting the reaction fluid

The reaction fluid was diluted in accordance with the following table.

TABLE 11

| Infectious viral titer in | 2.0 × 10⁴ | | . . . not counting in the reaction- |
|---|---|---|---|
| the reaction fluid = | (pfu/mL) | | induced titer reduction |
| Reaction fluid^{N.B.} | 100 | (μL) | N.B.: residual 100 μL was discarded |
| Serum-free MEM | 900 | (μL) | |
| Total fluid volume | 1000 | (μL) | . . . 10-fold diluted reaction fluid |
| ↓ | | | [10⁻¹] [symbol] used in plaque assay |
| 10-fold diluted reaction | 100 | (μL) | |
| fluid [10⁻¹] | | | |
| Serum-free MEM | 900 | (μL) | |
| Total fluid volume | 1000 | (μL) | . . . 100-fold diluted reaction fluid |
| ↓ | | | [10⁻²] [symbol] used in plaque assay |
| 100-fold diluted | 100 | (μL) | |
| reaction fluid [10⁻²] | | | |
| Serum-free MEM | 900 | (μL) | |
| Total fluid volume | 1000 | (μL) | . . . 1000-fold diluted reaction fluid |
| | | | [10⁻³] [symbol] usedin plaque assay |
| Reaction fluids | 500 | (μL) | |
| [10⁻¹], [10⁻²], [10⁻³] | | | |
| ↓ | | | |

Used in plaque assay

4.2. Plaque assay method

The MDCK cells were suspended in a MEM supplemented with 10% FBS and seeded on a 6-well plate to give a density of 1.0×10⁶ cells per well. The medium volume was so adjusted as to be 2.0 milliliters per well.

The seeded cells were cultured at 37° C. overnight in a 5% CO₂ atmosphere. Then, the broth was removed and the monolayer of cells was washed once with a serum-free MEM (2.0 milliliters per well). Subsequently, the diluted reaction fluids [10⁻¹], [10⁻²], [10⁻³] were added to each well to give a volume of 500 μL per well and the mixture was incubated at 37° C. for an hour in a 5% CO₂ atmosphere. Then, the reaction fluids were removed and the monolayer of cells was washed once with a serum-free MEM (2.0 milliliters per well). A 0.8% agarose containing maintenance medium (whose composition is shown in Table 12) was layered to give a volume of 3.0 milliliters per well and left to stand until the agarose solidified (in about 30 minutes). After culture was performed at 37° C. for 72 hours in a 5% CO₂ atmosphere, the emerging plaques were rendered visible by amido black staining and their number was counted.

TABLE 12
Composition of 0.8% Agarose
Containing Maintenance Medium

| Ingredients | inal concentration | |
|---|---|---|
| MEM | 1 | x |
| agarose | 0.8% | (w/v) |
| MEM vitamin | 1 | x |
| L-glutamine | 0.03% | (w/v) |
| BSA | 0.1% | (w/v) |
| Trypsin | 2.5 | μg/mL |

5. Results and Discussion

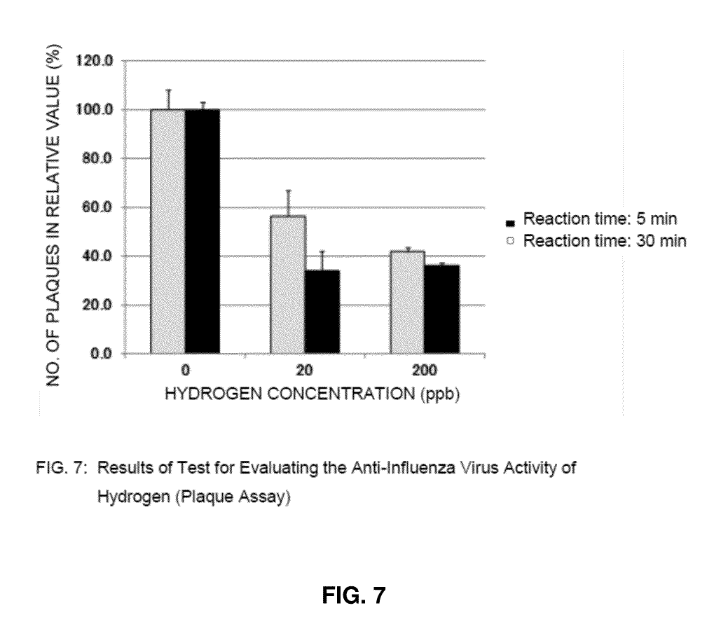

The results from the group treated with hydrogen for 30 minutes are shown in Table 13 (the number of emerging plaques and the numeral values obtained by analysis) and those from the group treated with hydrogen for 5 minutes are shown in Table 14 (the number of emerging plaques and the numeral values obtained by analysis). Those results were combined and shown graphically in FIG. 7.

TABLE 13
Reaction time = 30 min

| Hydrogen | | | |
|---|---|---|---|
| concentration (ppb) | 0 | 20 | 200 |
| No. of plaques | 69 | 28 | 25 |
| | 72 | 20 | 26 |
| Mean value | 70.5 | 24.0 | 25.5 |
| Standard deviation | 2.1 | 5.7 | 0.7 |
| Relative value (%) | 100.0 | 34.0 | 36.2 |
| Standard deviation (%) | 3.0 | 8.0 | 1.0 |

In the plaque forming test, the 1000-fold diluted reaction fluid [10⁻³] permitted plaque counting, so the number of plaques that emerged in the group of this reaction fluid, their mean value, and standard deviation are shown in Table 13. Relative values calculated with the number of plaques at the hydrogen concentration of 0 ppb (vehicle control group) being taken as 100% are also shown in Table 13.

TABLE 14
Reaction time = 5 min

| Hydrogen | | | |
|---|---|---|---|
| concentration (ppb) | 0 | 20 | 200 |
| No. of plaques | 81 | 42 | 35 |
| | 91 | 55 | 37 |
| Mean value | 86.0 | 48.5 | 36.0 |
| Standard deviation | 7.1 | 9.2 | 1.4 |
| Relative value (%) | 100.0 | 56.4 | 41.9 |
| Standard deviation (%) | 8.2 | 10.7 | 1.6 |

The number of plaques that emerged in the group undergoing 5-min reaction, their mean value, and standard deviation are shown in Table 14. Relative values calculated with the number of plaques at the hydrogen concentration of 0 ppb (vehicle control group) being taken as 100% are also shown in Table 14.

It was confirmed that when the influenza virus was treated with hydrogen for 5 minutes, the number of infectious particles was reduced to about 40% at maximum and by the 30-min treatment, a reduction to about 35% was achieved.

These results suggested that hydrogen has anti-viral activity against influenza A virus (Tamiflu-resistant strain).

### Example 8

### Test for Evaluating the Anti-RS Virus (Respiratory Syncytial Virus) Activity of Hydrogen

Materials

1. Hydrogen-containing preparation: SUISO PURE GOLD (ACCHE Corporation) was used. SUISO PURE GOLD (ACCHE Corporation) is a preparation that contains coral calcium, etc. having hydrogen molecules adsorbed thereon and which, upon contact with water, etc., generates hydrogen molecules, hydrogen ions (H⁺), hydride ions (H⁻) and atomic hydrogen (WO 2009/066463).

2. Viral strain: Strain A2 of the RS virus was used and amplified with HEp-2 cells derived from the ATCC (American Type Culture Collection) strain VR-1540.

Test for Evaluating the Cytotoxicity of Hydrogen and Test for Evaluating its Anti-RS Virus Activity (CPE Assay)

1. Method of preparing a hydrogen solution

A hydrogen solution was prepared by the same method as in Example 7.

2. Test Methods

Hydrogen's cytotoxicity was evaluated for HEp-2 cells; in addition, hydrogen's antiviral activity was evaluated for RS virus based on CPE (cytopathic effect) assay. Two controls for comparison were prepared; one of them was a group treated with physiological saline, i.e., the solvent in the hydrogen-containing preparation, and this was set as a vehicle control group, and another control was a group treated with the known antiviral agent ribavirin (Sigma Aldrich) which was set as a positive control group.

Specifically, using a MEM supplemented with 2% FBS, two-fold serial dilutions were prepared from the above-described hydrogen solution and the solvent (PS or physiological saline) (see the upper panel of Table 15). AdV was adjusted to 500 TCID50/mL with a 2% FBS supplemented MEM, mixed with an equal volume of the hydrogen solution in each well, and the mixture was stirred for 30 seconds. The final test concentrations of hydrogen in the liquid mixtures of virus and hydrogen are shown in the lower panel of Table 15.

The liquid mixtures of virus and hydrogen were left to stand at 37° C. for 5 minutes and then added in a volume of 0.2 milliliters/well to HEp-2 cells that had been seeded on the previous day. After 5 days of culture, the residual cells were stained by crystal violet staining and quantification was made by measuring the absorbance (OD at 560 nm) with a microplate reader.

TABLE 15

| Test Concentrations of Hydrogen and Vehicle Control | | | | | | | | | | | | | |
|---|---|---|---|---|---|---|---|---|---|---|---|---|---|
| (Physiological Saline) | | | | | | | | | | | | | |
| | Hydrogen concentration in 2-fold serial dilutions as prepared | | | | | | | | | | | | |
| Plate lane No. | 1 | 2 | 3 | 4 | 5 | 6 | 7 | 8 | 9 | 10 | 11 | 12 | |
| Conc. of H₂ | 0 | 0.39 | 0.78 | 1.56 | 3.13 | 6.25 | 12.5 | 25.0 | 50.0 | 100.0 | 200 | 400 | (ppb) |
| contained | | | | | | | | | | | | | |
| Conc. of PS | 0 | 0.10 | 0.20 | 0.39 | 0.78 | 1.56 | 3.13 | 6.25 | 12.5 | 25.0 | 50.0 | 100.0 | (%) |
| contained | | | | | | | | | | | | | |
| [symbol] | | | | | | | | | | | | | |
| | Hydrogen concentration after mixing with an equal volume of virus fluid = test concentration | | | | | | | | | | | | |
| Plate lane No. | 1 | 2 | 3 | 4 | 5 | 6 | 7 | 8 | 9 | 10 | 11 | 12 | |
| Conc. of H₂ | 0 | 0.20 | 0.39 | 0.78 | 1.56 | 3.13 | 6.25 | 12.5 | 25.0 | 50.0 | 100 | 200 | (ppb) |
| contained | | | | | | | | | | | | | |
| Conc. of PS | 0 | 0.05 | 0.10 | 0.20 | 0.39 | 0.78 | 1.56 | 3.13 | 6.25 | 12.5 | 25.0 | 50.0 | (%) |
| contained | | | | | | | | | | | | | |

Results

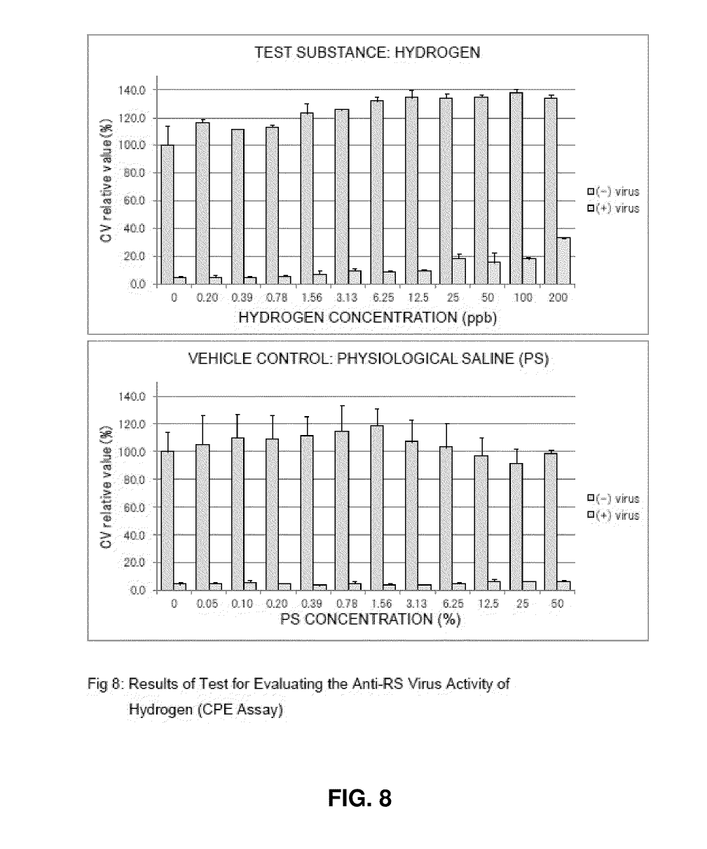

As the result of the cytotoxicity evaluation, hydrogen was not confirmed to have any marked toxicity to HEp-2 cells. As the result of the anti-viral activity evaluation by CPE assay, hydrogen was confirmed to show anti-viral activity against RS virus infection (FIG. 8). Using Graph Pad Prism 5.0 (manufactured by Graph Pad Software, Inc.), CC₅₀ (50% cytopathic concentration) IC₅₀ (50% infection inhibitory concentration) were calculated and shown in Table 16.

TABLE 16
Results of CC₅₀ and IC₅₀ Calculations

| | RS virus | | |
|---|---|---|---|
| | CC₅₀ | IC₅₀ | |
| Hydrogen | >200 | 2,708 | (ppb) |
| Ribavirin | >200 | 13.4 | (μM) |

### Example 9

### Test for Evaluating the Anti-AdV (Human Adenovirus) Activity of Hydrogen

Materials

1. Hydrogen-containing preparation: SUISO PURE GOLD (ACCHE Corporation) was used. SUISO PURE GOLD (ACCHE Corporation) is a preparation that contains coral calcium, etc. having hydrogen molecules adsorbed thereon and which, upon contact with water, etc., generates hydrogen molecules, hydrogen ions (H⁺), hydride ions (H⁻) and atomic hydrogen (WO 2009/066463).

2. Viral strain: Strain 5 of AdV was used; a clinically isolated strain 5 was amplified with HEp-2 cells derived from the ATCC (American Type Culture Collection) strain VR-1540.

Test for Evaluating the Anti-AdV Activity of Hydrogen (TCID₅₀ Assay)

1. Method of preparing a hydrogen solution

A hydrogen solution was prepared by the same method as in Examples 7 and 8.

2. Method of conditioning the virus fluid

The virus fluid was diluted with a 2% FBS supplemented MEM to be adjusted to 2.0×10⁵ TCID₅₀/mL.

3. Method of reaction between hydrogen and virus

The hydrogen solution prepared in 1 above was diluted with physiological saline to concentrations of 0, 40, and 400 ppb. The thus conditioned hydrogen solutions were each mixed with an equal volume of the virus fluid conditioned in 2 above; after reaction at 37° C. for 15, 30 and 60 minutes (Table 17), each reaction fluid was immediately diluted 10-fold with a 2% FBS supplemented MEM.

TABLE 17

| | Test Group No. | | | | | | | | | |
|---|---|---|---|---|---|---|---|---|---|---|
| | #0-15 | #20-15 | #200-15 | #0-30 | #20-30 | #200-30 | #0-60 | #20-60 | #200-60 | |
| 400 ppb H₂ solution | 0 | 25 | 250 | 0 | 25 | 250 | 0 | 25 | 250 | μL |
| Physiological saline | 250 | 225 | 0 | 250 | 225 | 0 | 250 | 225 | 0 | μL |
| Virus fluid (2.0 × 10⁵ pfu/mL) | 250 | 250 | 250 | 250 | 250 | 250 | 250 | 250 | 250 | μL |
| Total fluid volume | 500 | 500 | 500 | 500 | 500 | 500 | 500 | 500 | 500 | μL |
| Test substance's | 0 | 20 | 200 | 0 | 20 | 200 | 0 | 20 | 200 | |
| concentration (ppb) | | | | | | | | | | |

| | | | |
|---|---|---|---|
| Reaction time | 5 min. | 30 min. | 60 min |

| | |
|---|---|
| Reaction temperature | 37° C.^{N.B.} |
| N number | 2 (duplicate) |

N.B.: A thermostated incubator was used.
Total number of groups = 18 groups/virus

4. Method of TCID₅₀ assay

A 10-fold dilution of the reaction fluid prepared in 3 above was added to a 96-well plate for serial dilution and a total of eight 10-fold serial dilutions (10⁻¹-10⁻⁵) were prepared. HEp-2 cells that had been seeded on a 96-well plate were freed of the culture supernatant and the 10-fold serial dilutions of the reaction fluid were each added in a volume of 200 μL/well. After 5-day culture under the 37° C./5% CO₂ condition, crystal violet staining was performed to check for the occurrence of any infection and the infectious viral titer per unit volume was calculated in accordance with the Reed & Munch formula.

5. Results and Discussion

The viral titer of AdV as calculated at the hydrogen concentration of 200 ppb was found to decrease to one tenth of the value for the untreated group after the 30-min reaction, and to less than a hundredth after the 60-min reaction. The calculated values of infectious viral titer (mean on duplicates) are shown in Table 18, and the relative values with the titer for the hydrogen-free group after the respective reaction times being taken as 100% are shown in Table 19.

TABLE 18
TCID50 Assay Results
(indicating calculated titers)

| Hydrogen | | | |
|---|---|---|---|
| concentration | Reaction time | | |
| (ppb) | 15 min | 30 min | 60 min |
| 0 | 1580 | 1580 | 1580 |
| 20 | 1580 | 1580 | 1580 |
| 200 | 158 | 158 | ≦50 |

*Unit is TCID₅₀/mL

From the results of the TCID₅₀ assay, infectious viral titers were calculated in accordance with the Reed & Munch method. Each test was conducted with N=2 (duplicate) and the numerical values in the table are their means.

TABLE 19
TCID₅₀ Assay Results
(indicating relative values)

| Hydrogen | | | |
|---|---|---|---|
| concentration | Reaction time | | |
| (ppb) | 15 min | 30 min | 60 min |
| 0 | 100% | 100% | 100% |
| 20 | 100% | 100% | 100% |
| 200 | 10% | 10% | ≦3% |

For conversion to relative values, the titer for the hydrogen-free group (0 ppb) after the respective reaction times was taken as 100%.

As described above, AdV (adenovirus), upon reaction with 200 ppb hydrogen, was found to be attenuated in infectious viral titer in a reaction-time dependent manner until it dropped below the detection limit after the passage of 60 minutes. Thus, the results of anti-virus activity evaluation by TCID₅₀ assay verified that hydrogen displays anti-viral activity against AdV infection.

1-26. (canceled)

27. An antimicrobial agent comprising: at least one member selected from the group consisting of hydrogen atom (H); at least one hydrogen isotope selected from the group consisting of hydrogen atom (H); ¹H, ²H, ³H, ⁴H, ⁵H, ⁶H and ⁷H; hydrogen molecule (H²); a metal hydride; hydrogen ion (H⁺); hydride ion (H⁻); and atomic hydrogen.

28. The antimicrobial agent according to claim 27, which contains a coral powder having the hydrogen molecule adsorbed thereon.

29. The antimicrobial agent according to claim 27, wherein the hydrogen molecule or metal hydride ionizes upon contact with water to generate a hydride ion.

30. The antimicrobial agent according to claim 27, wherein the metal hydride is a hydride of a metal of at least one species selected from Group 1, Group 2, Group 13 or Group 14 in the periodic table of elements.

31. The antimicrobial agent according to claim 30, wherein the metal hydride comprises calcium hydride.

32. The antimicrobial agent according to claim 27, which contains a reduction fired body of mollusk shell, livestock's bone, fish bone, calcified coral, coral calcium, calcium carbonate, silica, zeolite, or two or more combinations thereof.

33. The antimicrobial agent according to claim 27, wherein the fungus is a yeast-like fungus.

34. The antimicrobial agent according to claim 33, wherein the fungus is at least one yeast-like fungus selected from the group consisting of Candida albicans and Candida glabrata.

35. The antimicrobial agent according to claim 27, wherein the virus is at least one virus selected from the group consisting of viruses belonging to the family Orthomyxoviridae, viruses belonging to the family Paramyxoviridae, and viruses belonging to the family Adenoviridae.

36. The antimicrobial agent according to claim 35, wherein the virus is at least one virus selected from the group consisting of influenza viruses, RS viruses, and adenoviruses.

37. The antimicrobial agent according to claim 35, wherein the virus is an amantadine-resistant influenza virus or Tamiflu-resistant influenza virus.

38. The antimicrobial agent according to claim 27, which is in a gaseous form.

39. The antimicrobial agent according to claim 27, which is in an aerosol form.

40. The antimicrobial agent according to claim 27, which is in a liquid form.

41. The antimicrobial agent according to claim 27, which is in a solid form.

42. The antimicrobial agent according to claim 41, which is powdery.

43. The antimicrobial agent according to claim 42, which provides an approximate concentration of hydrogen when three grams of the antimicrobial agent is mixed with 20 milliliters of physiological saline.

44. A pharmaceutical composition containing the antimicrobial agent according to claim 27.

45. The pharmaceutical composition according to claim 44, which is to be used in combination with at least one other medication.

46. A microorganism control agent containing the antimicrobial agent according to claim 27.

47. A method for preventing or managing bacterial infection, comprising:
administering to a subject in need thereof an effective amount of an antimicrobial agent comprising: a coral powder containing the hydrogen molecule adsorbed thereon.

48. The method according to claim 47, which is for preventing or managing infection with at least one bacterium belonging to Gram-negative bacilli, Gram-negative cocci, Gram-positive cocci, or Gram-positive bacilli.

49. A method for preventing or managing bacterial infection, comprising:
administering to a subject in need thereof an effective amount of an antimicrobial agent comprising: a reduction fired body of mollusk shell, livestock's bone, fish bone, calcified coral, coral calcium, calcium carbonate, silica, zeolite, or two or more combinations thereof.

50. The method according to claim 49, wherein the bacterium is at least one bacterium selected from the group consisting of Escherichia coli, pathogenic Escherichia coli O157, Salmonella, Haemophilus influenzae, Vibrio parahaemolyticus, Enterococcus, Pneumococcus, Neisseria, Neisseria gonorrhoeae, Neisseria meningitidis, Staphylococcus aureus, Staphylococcus epidermidis, Group A Streptococcus, Group B Streptococcus, Group C/G Streptococcus, Listeria monocytogenes, Klebsiella pneumoniae, Shigella, Vibrio cholerae, B. cepacia, Citrobacter, and Serratia.

51. The method according to claim 49, wherein the bacterium is at least one bacterium selected from the group consisting of extended-spectrum β-lactamase (ESBL) producing Gram-negative bacilli, multidrug-resistant Pseudomonas aeruginosa (MDRP), New Delhi metallo-β-lactamase (NDM-1) producing Gram-negative bacilli, β-lactamase non-producing ampicillin-resistant (BLNAR) Haemophilus influenzae, methicillin-resistant Staphylococcus aureus (MRSA), vancomycin-resistant Enterococcus (VRE), penicillin-resistant Streptococcus pneumoniae (PRSP), multidrug-resistant Acinetobacter (MDRA), Klebsiella pneumoniae carbapenemase producing bacterium (KPC), penicillinase-producing Neisseria gonorrhoeae (PPNG) and community-acquired infection type methicillin-resistant Staphylococcus aureus (CA-MRSA).
